SEQUENCE LISTING (1 sequence)

SEQ ID NO: 1 - DNA, 35 bp, Artificial Sequence
  Synthetic polynucleotide
caggctacggcacgtagagcatcaccatgatcctg

(19) United States
(12) Patent Application Publication
(10) Pub. No.: US 2014/0031253 A1
(43) Pub. Date: Jan. 30, 2014

(54) Protein Detection Using Three-Dimensional Carbon Microarrays

(71) Applicants: Varun Penmatsa, Miami, FL (US); Chunlei Wang, Miami, FL (US); Hiroshi Kawarada, Tokyo (JP)

(72) Inventors: Varun Penmatsa, Miami, FL (US); Chunlei Wang, Miami, FL (US); Hiroshi Kawarada, Tokyo (JP)

(73) Assignee: THE FLORIDA INTERNATIONAL UNIVERSITY BOARD OF TRUSTEES, Miami, FL (US)

(21) Appl. No.: 13/946,810

(22) Filed: Jul. 19, 2013

Related U.S. Application Data
(60) Provisional application No. 61/676,383, filed on Jul. 27, 2012.

(51) Int. Cl. G01N 33/551; G01N 33/74

(52) U.S. Cl. CPC G01N 33/551; G01N 33/74
USPC 506/9; 506/16

(57) ABSTRACT

The potential of aptamers as ligand binding molecule have opened new avenues in the development of biosensors for proteins, such as cancer oncoproteins. Disclosed herein is a label-free detection strategy using signaling aptamer/protein binding complex for proteins, such as platelet-derived growth factor (PDGF-BB) oncoprotein. The detection mechanism is based on the release of a fluorophore (e.g., TOTO intercalating dye) from the target binding aptamer's stem structure when it captures the protein, e.g., PDGF. Amino-terminated three-dimensional carbon microarrays fabricated by pyrolyzing patterned photoresist are used as a detection platform. The sensor showed near linear relationship between the relative fluorescence difference and protein concentration even in the sub-nanomolar range with an excellent detection limit of 5 pmol. This detection strategy is promising in a wide range of applications in the detection of cancer biomarkers and other proteins.

### BACKGROUND

With the increasing application of proteomic strategies for the detection of cancer related oncoproteins and discovery of biomarkers, it is of interest to develop portable platforms for sensitive detection of proteins and their molecular variants. Aptamers are single stranded DNA or RNA molecules selected in vitro from DNA/RNA random pools that are capable of binding with biological entities such as proteins, cells along with small molecules, drugs, peptides and hormones with high affinity and specificity (Ellington and Szostak, 1990; Robertson and Joyce, 1990; Tuerk and Gold, 1990). Aptamers have been sought out as alternative candidates to the traditional antibodies for use in analytical devices due to their easy synthesis, high binding affinity, long storage times, and excellent selectivity (Jayasena, 1999). Recent studies have demonstrated the applicability of aptamers to target a disease state, such as cancer (Shangguan et al., 2006). This opens up new avenues in the future for aptamers to potentially substitute more established components for therapeutics and/or diagnostics.

Platelet-derived growth factor (PDGF) is a protein that regulates cell growth and division. Overexpression of PDGF has been associated with several human health disorders including atherosclerosis (hardening of the arteries) (Lassila et al., 2004), balloon injury induced restenosis (narrowing of blood vessels) (Szabo et al., 2007), pulmonary hypertension (Barst, 2005), organ fibrosis (formation of excess fibrous connective tissue in an organ or tissue) (Trojanowska, 2008), tumorigenesis (formation of tumors) (Shih et al., 2004). PDGF receptors are almost undetectable in normal vessels, but are highly expressed in the diseased vessels. A PDGF dimer composed of two different types of monomer (A and B chains) occurs in three variants: PDGF-BB, PDGF-AB and PDGF-AA. In particular, oncoprotein PDGF-BB is often overexpressed in human malignant tumors and known as a potential protein marker for cancer diagnosis (Shih et al., 2004).

In recent years, PDGF-BB protein detection using fluorescence (Yang et al., 2007; Ruslinda et al., 2012; Fang et al., 2001; Fang et al., 2003; Vicens et al., 2005; Yang et al., 2005, Jiang et al., 2004; Zhou et al., 2006; Huang et al., 2007; Huang et al., 2008) colorimetry (Huang et al., 2005) and electrochemistry techniques have been reported (Lai et al., 2007; Degefa and Kwak, 2008; Ruslinda et al., 2010). These methods involve either labeling the aptamer with a fluorophore, or the use of redox species. In fluorescence based PDGF detection techniques, fluorophore-labeled aptamers are used to signal binding by monitoring the changes of fluorescence intensity (Fang et al., 2003) or anisotropy resulting from the changes of the microenvironment (Fang et al., 2001) or rotational motion through fluorescence energy transfer (Vicens et al., 2005). However, as the precise target binding sites and the conformational changes of the aptamers are generally unknown, it is not easy to design labeling strategies. Additionally, there is a concern that the conjugation of a fluorophore to an aptamer will weaken the affinity of the aptamer to its ligand (Huang et al., 2007). In the case of electrochemistry based detection techniques, due to the use of redox species, the electrodes are limited to conductive materials and also the different linkers used to attach the aptamer onto the electrode surface (such as gold) exhibits rapid degradation with time (Phillips et al., 2008). Most recently, diamond substrate has been used to detect PDGF by monitoring the fluorescence change from the release of an intercalating dye when the probe aptamer captures the target (Ishii et al., 2011). Although the sensor showed good sensitivity and selectivity, the use of diamond substrates is not cost effective. The controllability of defects and grain boundaries in polycrystalline diamond substrates along with the high operating cost due to the need for high vacuum and high temperature systems are limiting factors for mass production.

For biological and electrochemical sensing, glassy carbon is often used due to its low cost, better resistance towards biofouling, biocompatibility, good electrical conductivity, low background capacitance, and the flexibility to tailor the surface by various physical/chemical treatments. In particular, carbon synthesized by carbon-microelectromechanical systems (C-MEMS) technique (also known as pyrolyzed photoresist carbon), where organic photoresist patterns are heat treated at high temperatures and oxygen free environment, is intriguing since it exhibits reaction kinetics comparable to glassy carbon, but with lower oxygen/carbon atomic (O/C) ratio (Wang et al., 2005; Ranganathan et al., 2000, Singh et al., 2000). Since photolithography is used for patterning purpose, the electrodes obtained by this manner have better resolution and reproducibility compared to screen printed carbon paste electrodes. C-MEMS technique is actively pursued to fabricate electrodes for DNA biosensors (Yang et al., 2009), glucose sensors (Xu et al., 2008), protein detection (Lee et al., 2008), microbatteries (Wang et al., 2004) and on-chip supercapacitors (Chen et al., 2010) due to the versatility in the experimental approach to produce high surface area 3D carbon microarrays. In addition, the ability to tailor the carbon surface is possible by introducing nanoporosity using a block copolymer as porogen (Penmatsa et al., 2010) and integration of functional nanomaterials such as graphene (Penmatsa et al., 2012) and carbon nanotubes on the surface of 3D carbon microarrays (Chen et al., 2010).

The high surface area of the 3D carbon microarrays makes it an ideal platform for increased biomolecule loading to improve the sensitivity and performance of the functional devices.

### SUMMARY

In one aspect, described herein is a method for detection of a biomarker in a biological sample, the method comprising contacting the biological sample with sensor that comprises an aptamer immobilized on a substrate, wherein the aptamer that selectively binds to the biomarker; and wherein the substrate comprises a three dimensional (3D) carbon microarray; and wherein the substrate is not a diamond substrate; and detecting the biomarker in the biological sample by detecting biomarker bound to the sensor. In some embodiments, the 3D carbon microarray comprises pyrolyzed photoresist carbon.

In some embodiments, the method further comprises measuring the biomarker in the biological sample by measuring the amount of biomarker bound to the sensor. The measuring of the biomarker optionally comprises measuring a fluorescence signal from the dye, which is indicative of the amount of the biomarker bound to the aptamer.

The aptamer optionally comprises the oligonucleotide set forth in SEQ ID NO: 1. In some embodiments, the aptamer further comprises an intercalating due, such as dye 1,1-(4,4,8,8-tetramethyl-4,8-diazaundecamethylene)-bis-4-[3-methyl-2,3-dihydro(benzo-1,3-thiazole)-2-methylidene]quinolinium tetraiodide (TOTO). The aptamer is optionally covalently attached to the substrate (e.g., by an amide bond without the use of a linker molecule). In some embodiments, the aptamer comprises a carboxyl modified aptamer and the substrate comprises a 3D carbon microarray by direct amination.

The biomarker in the biological sample can be any protein. In some embodiments, the biomarker is a growth factor protein. In some embodiments, the growth factor protein comprises a platelet derived growth factor (PDGF) protein, such as PDGF-A or PDGF-B.

In some embodiments, the growth factor is present in the biological sample as a dimer. For example, in some embodiments, PDGF is present in the biological sample as a dimer, such as PDGF-AA, PDGF-AB or PDGF-BB.

The biomarker, in some embodiments, is suspected of being present in the biological sample as a sub-nanomolar concentration. In some embodiments, the biomarker is present in the sample at a concentration of less than 1 nM. In other embodiments, the biomarker is present in the sample at a concentration of between 0.005 nM and about 100 nM.

In some embodiments, the biological sample comprises blood, serum or plasma. The biological sample is preferably from a human subject. In some embodiments, the human subject has a disorder selected from the group consisting of cancer, atherosclerosis, balloon injury induced restenosis, pulmonary hypertension, organ fibrosis and tumorigenesis. In some embodiments, the human subject has a cancer associated with PDGF-B.

In another aspect, described herein is a sensor comprising a three-dimensional (3D) carbon microarray substrate comprising pyrolyzed photoresist carbon; and an aptamer covalently attached to the substrate, wherein the aptamer selectively binds a biomarker and wherein the aptamer further comprises a intercalating dye, such as dye 1,1-(4,4,8,8-tetramethyl-4,8-diazaundecamethylene)-bis-4-[3-methyl-2,3-dihydro(benzo-1,3-thiazole)-2-methylidene]quinolinium tetraiodide (TOTO). In some embodiments, the aptamer is a carboxyl modified aptamer and the substrate is modified by direct amination. The aptamer is optionally covalently attached to the substrate by an amide bond without the use of alinker molecule.

In some embodiments, the aptamer comprises an oligonucleotide, such as the oligonucleotide set forth in SEQ ID NO: 1.

The sensor is useful for the detection of a biomarker in a biological sample. In some embodiments, the sensor detects the presence of a biomarker in a biological sample at a concentration of less than 1 nM or at a concentration ranging from about 0.005 nM to about 100 nM.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicant(s) by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIG. 1: (a) Typical SEM image of 3D carbon microarrays, (b) Raman spectrum of pyrolyzed photoresist film showing the two prominent bands at 1350 and 1590 cm⁻¹.

FIG. 2: Deconvoluted C1s spectra of pyrolyzed photoresist film after 4 hr direct amination, here dash line shows the original data and solid lines show the fitting curves. Inset shows the widescan XPS spectra of carbon film before and after amination.

FIG. 3: Schematic illustration of the detection of PDGF-BB using signaling aptamer/protein binding complex on 3D carbon microarrays platform; (I) covalent immobilization of PDGF-binding aptamer on partially aminated carbon surface, (II) intercalating the probe aptamer with TOTO fluorescent dye, (III) binding PDGF-BB to the aptamer-intercalating dye complex, (IV) regenerating the sensor by sodium dodecyl sulfate (SDS) treatment to remove PDGF and release the intercalating dye.

FIG. 4: (a) Relative fluorescence difference response of the sensor to different concentrations of PDGF from 0.005 nM to 100 nM. The concentrations of the aptamer and intercalating dye were 20 μM and 10 μM, respectively, (b) Comparison of relative fluorescence difference of different proteins towards PDGF binding aptamer; The concentration of the different molecules (PDGF-BB, PDGF-AB, PDGF-AA, BSA, ATP and calmodulin) was 100 nM and concentrations of PDGF-binding aptamer and intercalating dye were 20 μM and 10 μM, respectively.

### DETAILED DESCRIPTION

Disclosed herein is a signaling aptamer/protein binding complex on 3D carbon micropillar arrays using TOTO intercalating dye to signal PDGF-BB-aptamer binding. The carbon surface was functionalized by direct amination technique to introduce amino groups for covalent immobilization of target binding aptamer. This simple detection technique offers high sensitivity with PDGF detection in the sub-nanomolar range and good selectivity against different proteins, which can be extended for the detection of other biomarker proteins. Modification of the specific methods disclosed below for other biomarker proteins comprise using aptamers labeled with a dye, specific for the desired biomarker protein.

In general, aptamers are nucleic acid or peptide binding species capable of tightly binding to and discreetly distinguishing target ligands [Yan et al., RNA Biol. 6(3) 316-320 (2009), incorporated by reference herein in its entirety]. Aptamers, in some embodiments, may be obtained by a technique called the systematic evolution of ligands by exponential enrichment (SELEX) process [Tuerk et al., Science 249:505-10 (1990), U.S. Pat. No. 5,270,163, and U.S. Pat. No. 5,637,459, each of which is incorporated herein by reference in their entirety].

U.S. Pat. No. 5,637,459 describes the SELES process as having the following steps:

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: a) to assist in the amplification steps described below; b) to mimic a sequence known to bind to the target; or c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5-50%) are retained during partitioning.

4) Those nucleic acids selected during partitioning as having the relatively higher affinity to the target are then amplified to create a new candidate mixture that is enriched in nucleic acids having a relatively higher affinity for the target.

5) By repeating the partitioning and amplifying steps above, each successively formed candidate mixture contains fewer and fewer unique sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

The SELEX Patent Applications describe and elaborate on this process in great detail. Included are targets that can be used in the process; methods for the preparation of the initial candidate mixture; methods for partitioning nucleic acids within a candidate mixture; and methods for amplifying partitioned nucleic acids to generate enriched candidate mixtures. The SELEX Patent Applications also describe ligand solutions obtained to a number of target species, including protein targets wherein the natural role of the protein is and is not a nucleic acid binding protein. For example, in U.S. Pat. No. 5,496,938 (which is incorporated herein by reference in its entirety) methods are described for obtaining improved nucleic acid ligands after SELEX has been performed.

In addition, general discussions of nucleic acid aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2(1): 72-79 (2003). In various aspects, an aptamer is between 10-100 nucleotides in length. The aptamer specific for a biomarker of interest can be attached to the 3D carbon micropillar array as disclosed herein in a similar manner as that described for PDGF specifically.

### EXAMPLES

### Example 1

### Fabrication of 3D Carbon Microarrays

The three-dimensional carbon microarrays were fabricated by a typical C-MEMS process. 4 in. silicon oxide wafers were spin cleaned by acetone and methanol followed by a 5 minutes bake on the hotplate at 150° C. for 5 minutes to evaporate any moisture. NANO™ SU-8 100 negative photoresist was spin coated using a photoresist spinner (Headway Research™) at 500 rpm for 12 sec and then 1200 rpm for 30 seconds to get approximately 200 μm photoresist film. The photoresist was baked at 65° C. for 10 minutes and at 95° C. for 30 minutes in order to harden the photoresist by evaporating any remaining solvents. The photoresist was patterned by exposure using OAI Hybralign contact aligner (light intensity, 17 mW/cm²) for 60 sec to crosslink polymer chains in the photoresist. Post expose bake was carried out at temperatures of 65° C. for 1 minutes and 95° C. for 3 minutes respectively to further harden the crosslinked photoresist. The patterned samples were developed using NANO™ SU-8 developer (Microchem, USA) for 15 minutes to wash away the unwanted photoresist. The pyrolysis of the photoresist microarrays was conducted in a Lindberg tube furnace under (95% N₂+5% H₂) environment. The samples were heated from room temperature to 350° C. at 2° C./minute rate with a hold time of 40 min, followed by ramping to 1000° C. at 5° C./minute rate and hold time of 60 min. The samples were cooled down under to room temperature in the inert atmosphere.

### Example 2

### Surface Functionalization Using Direct Amination

Before the direct amination process, the samples were first thoroughly rinsed with DI water and blow dried. The amination process was performed at room temperature in an ammonia gas (99.9%) environment and using UV lamp (wavelength=253.7 nm). Prior to UV irradiation, the reaction chamber was purged with nitrogen gas for 5 minutes to remove oxygen and other gases. The reaction chamber was then irradiated with UV light for 4 hr under a continuous flow of ammonia gas at 100 sccm. Finally, nitrogen gas is purged for 5 minutes to remove any ammonia in the reaction chamber before removing the sample.

### Example 3

### Detection of the Protein

The 5′-carboxyl-modified PDGF-B-binding aptamer (5′-CAG GCT ACG GCA CGT AGA GCA TCA CCA TGA TCC TG-3′) (SEQ ID NO: 1), PDGF-BB, PDGF-AB, PDGF-AA, adenosine triphosphate (ATP), and calmodulin were purchased from Sigma Genosys. The intercalating dye 1,1-(4,4,8,8-tetramethyl-4,8-diazaundecamethylene)-bis-4-[3-methyl-2,3-dihydro(benzo-1,3-thiazole)-2-methylidene]quinolinium tetraiodide (TOTO) was purchased from Invitrogen Corporation. The carboxyl modified PDGF-B aptamer was covalently immobilized on the amino-terminated carbon surface without the use of any linker molecules. The probe aptamer with 3× sodium saline citrate (SSC) buffer solution, 0.1 M N-hydroxysuccinimide (NHS) and 0.4 M 1-ethyl-3-(3-dimethylaminopropyl) carbodiimide hydrochloride (EDC) were mixed in a 2:1:1 ratio. The final concentration of the probe aptamer solution was 20 μM. 5 μM of the probe aptamer solution (probe aptamer concentration is 20 μM) was dropped onto the 3D carbon microarrays and incubated for 2 hours at 38° C. in a humidified chamber. After immobilization, the sample was washed in PBS+Tween-20 (PBS: 1 mM NaCl₂ mM NaH₂PO₄: 8 mM Na₂HPO₄; 0.1% Tween-20) solution for 5 minutes and three times with deionized (DI) water for 3 minutes each. The probe aptamer was then reacted with 10 μM intercalating dye (TOTO) diluted in TE buffer [10 mM tris(hydroxymethyl)-aminomethane (Tris), 1 mM ethylenediaminetetraacetic acid (EDTA), pH ˜8] for 1 hour at 25° C. Following the intercalation of the dye, the sample was cleaned by TE buffer for 20 minutes and a DI water rinse. PDGF-BB protein diluted in 2×SSC was then bound to the immobilized aptamer at room temperature for 1 hour at 25° C. Unbound PDGF-BB were cleaned by DI water for 5 minutes. It is noteworthy that certain monovalent and divalent cations commonly encountered in biological specimens are known to affect DNA conformation. For this reason, we selected the concentrations of the solution based on our previous study concerning the effect of protein binding based on Mg²⁺ cation and NaCl concentration in PBS buffer solution.²⁸ Finally, in order to regenerate the sensor by dissociating PDGF-BB and intercalator from the probe aptamer, the sample is washed in 10% sodium dodecyl sulfate (SDS) solution for 30 minutes.

### Example 4

### Characterization

The morphology of 3D carbon microarrays was investigated using JOEL 6335 FE-SEM scanning electron microscopy. Raman spectrum was collected with an argon ion laser system (Spectra Physics, model 177G02) of λ=514.5 nm at a laser power of ca. 7 mW. The chemical composition of pyrolyzed photoresist carbon film before and after dlirect amination procedure was investigated by an Ulvac Φ 3300 x-ray photoelectron spectroscopy (XPS) with an anode source providing Al Kα radiation. The electron takeoff angle was 45±3° relative to the substrate surface. Fluorescence observation was performed using an Olympus IX71 epifluorescence microscope.

### Discussion

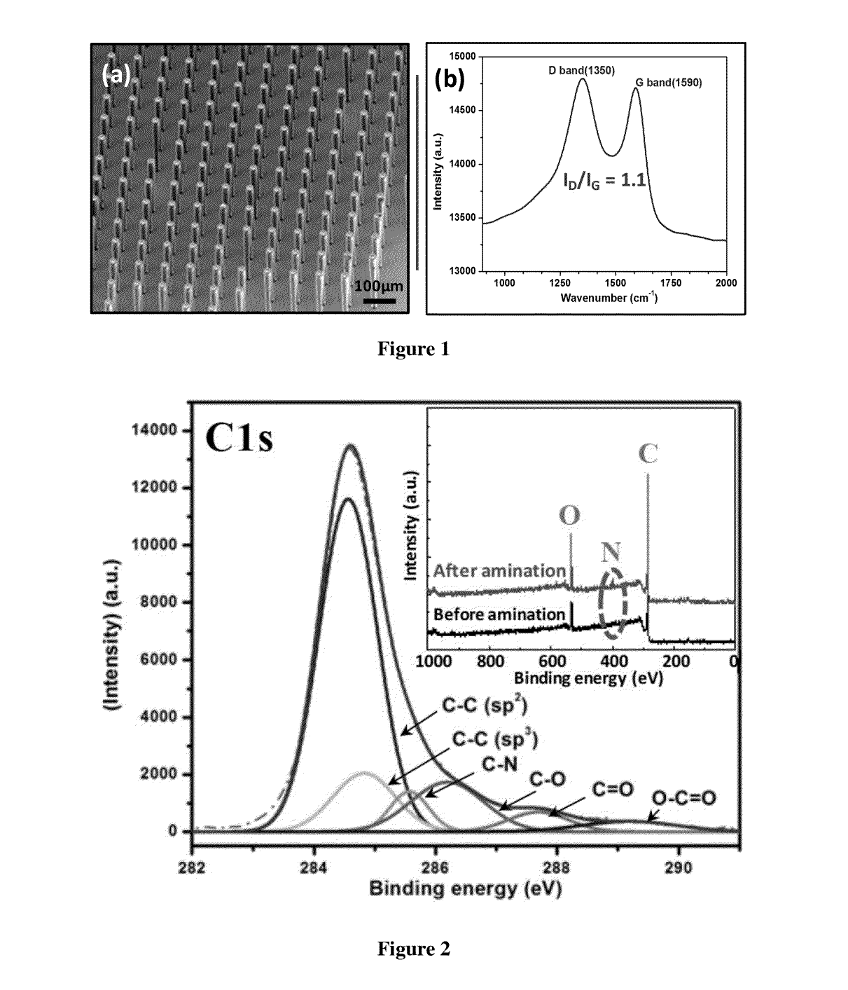

A typical SEM image of high aspect ratio 3D carbon micropillar arrays is shown in FIG. 1A. The average dimensions of the carbon micropillars after carbonizing patterned SU-8 photoresist structures are ˜160 μm height and ˜30 μm width. A careful examination of the SEM image shows that the upper half and especially the top part of the carbon micropillars is slightly wider compared to the lower half, which can be attributed to the higher dose of UV light experienced by the top layer of the thick photoresist (Wang et al., 2005). Raman spectroscopy was used to investigate the crystallinity of the carbon micropillars. FIG. 1B shows the Raman spectrum of pyrolyzed carbon with two significant broad peaks at ˜1350 cm⁻¹ (D-band) and ˜1590 cm⁻¹ (G-band). The first peak at 1350 cm⁻¹ represents the disorder band of the microcrystallite graphite and the second peak at 1590 cm⁻¹ is due to the single Raman line typically found on single crystalline graphite. The I_{D}/I_{G} ratio of 1.1 indicates that carbon obtained from pyrolysis of photoresist is identical to glassy carbon synthesized at same temperature (Penmatsa et al., 2012).

It is well documented that the termination or functionalization of the surface is one of the key issues in the interaction and immobilization of biomolecules (Kawarada and Ruslinda, 2011). In this work, to covalently immobilize PDGF binding aptamer on the carbon surface, the sample was first treated by direct amination technique (Yang et al, 2009) where the sample was irradiated by ultraviolet (UV) light (λ=253.7 nm) in an ammonia gas environment for 4 hrs. In contrast to oxidation techniques which introduce several oxygen-based functional groups such as ketone, hydroxyl, and carboxyl groups, only NH₂ bonds are expected to form on the carbon surface by direct amination procedure due to their chemical structure. The elemental composition and surface binding of pyrolyzed photoresist film were evaluated by X-ray photoelectron spectroscopy spectra (XPS) as shown in FIG. 2. Analysis of the wide can XPS spectra of the bare carbon film before amination (FIG. 2 inset) shows two major peaks evident of carbon (284.6 eV) and oxygen (531.8 eV) but in the case of after amination, three distinct peaks representing carbon, oxygen and nitrogen (398.4 eV) are evident. The nitrogen peak visible after amination is a result of ammonia gas forming C—NH₂ on the carbon substrate. The deconvoluted high resolution C1s spectrum (FIG. 2) shows major carbon peaks at 284.6 eV (sp²) and 285.2 eV (sp³), respectively. The other peaks at 285.4, 286.3, 287.6 and 289.1 eV corresponds to C—N, C—O, C═O and O—C═O bonds, respectively. The maximum surface coverage of amino groups achieved was ˜8%, which is similar to the amino coverage previously reported (Yang et al, 2009).

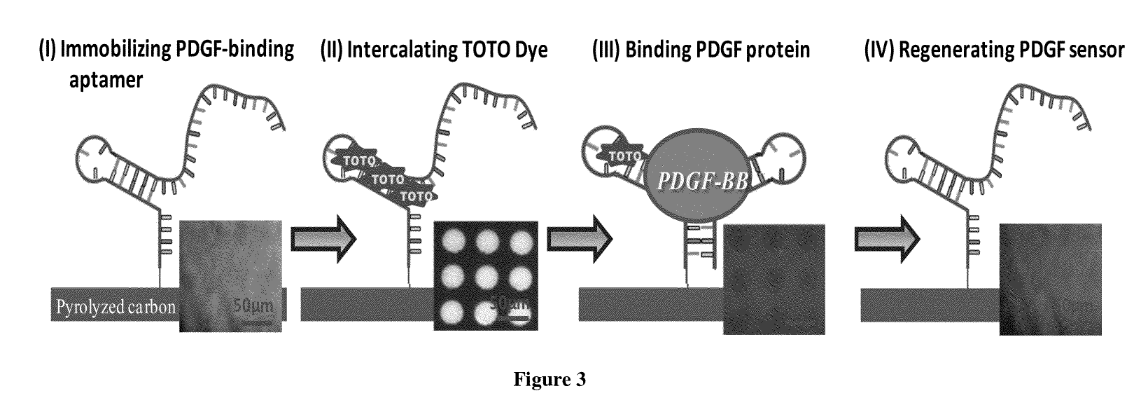

The detection of PDGF-BB using signaling aptamer/protein binding complex strategy is shown schematically in FIG. 3. (I) The carboxyl-terminated PDGF-binding aptamer (probe aptamer) is first covalently attached to the amine-terminated carbon surface via amide binding. (II) Subsequently the TOTO dye was intercalated with PDGF-binding aptamer. The TOTO dye shows no fluorescence in aqueous solution but exhibits strong fluorescence when bound to the nonaqueous pocket of the duplex nucleic acid regions in the aptamer. It is important to note that the fluorescence signal from TOTO is dependent on its local environment and DNA/RNA conformation. (III) When the target PDGF-BB protein bonds with the aptamer, the induced conformational change of the aptamer, as well as the blocking of intercalated TOTO dye results in a significant protein-dependent fluorescence change. (IV) Finally for regenerating the sensor, the aptamer intercalating dye complex and PDGF-BB are dissociated by treatment with sodium dodecyl sulfate (SDS).

The relationship of the change in the relative fluorescence difference with different concentrations of PDGF-BB in 2×SSC (saline-sodium citrate) solution was evaluated to study the sensitivity of the sensor. At first, the difference in the fluorescence intensity values is computed from the fluorescence intensity values obtained after initial TOTO intercalation with the probe aptamer and then after PDGF-BB binding with the probe aptamer. Then, the relative fluorescence difference is calculated by dividing the value obtained from difference in fluorescence intensities and initial fluorescence intensity. As expected, analysis of the data shows that the relative fluorescence difference increased as the concentration of PDGF-BB was increased from 0.005-100 nmol. This can be explained by the fact that, as the PDGF-BB concentration is increased, more intercalator dye is released from the aptamer which results in a larger difference in the relative fluorescence. A near linear relationship between the relative fluorescence difference and the protein concentration was observed even in the sub-nanomolar range. A low detection limit of 0.005 nmol was achieved, and indicates that the sensor detection limit is much below the typical detection range of the PDGF in clinical samples. The detection limit by other reported aptamer-based analytical techniques, for example, is 1 nmol in undiluted serum and 0.05 nmol in 50% serum was achieved with electrochemical detection (Lai et al., 2007), 0.1 nmol using solution based fluorescent signaling complex of aptamer and TOTO (Fang et al., 2001), and 2 nmol with fluorescence anisotropy based detection (Fang et al., 2001). Typical PDGF concentrations of normal individuals and cancer patients have been found to be in the sub-nanomolar range: 0.4-0.7 nmol in human blood serum and 0.008-0.04 nmol in human plasma (Ruslinda et al., 2012). Therefore, with the excellent sensitivity we achieved, we expect that this PDGF sensor has the potential to be used in clinical setting.

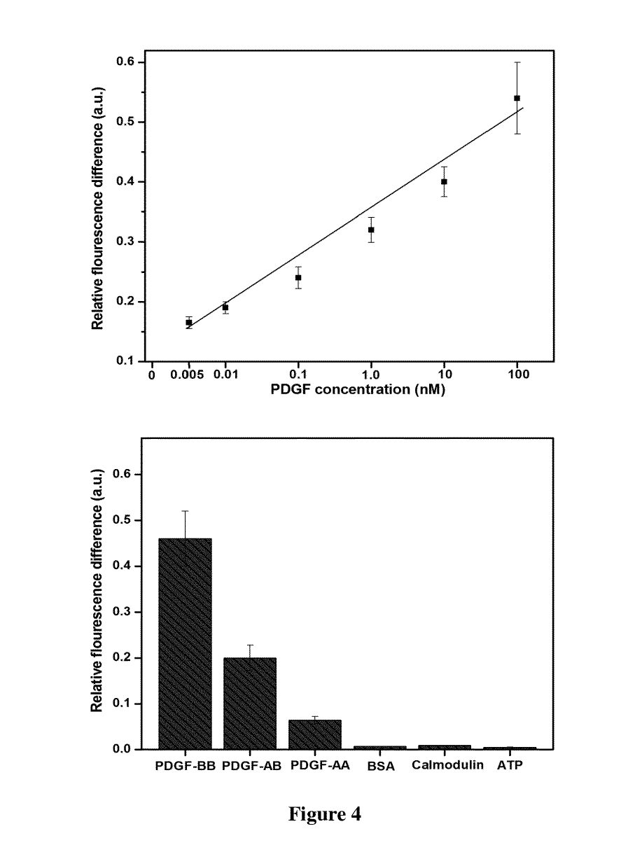

After the regeneration of the same sensor platform, in order to detect the PDGF using the aptamer based sensor, the probe should selectively respond to PDGF-BB, free or distinguishable from the interference by other biological components. FIG. 4b shows the selectivity test of PDGF binding aptamer towards the three variants of PDGF along with bovine serum albumin, calmodulin, and ATP, which are all typically present in the blood. The graph shows that the relative fluorescence difference for PDGF-BB binding with probe aptamer was about two times that of PDGF-AB and 10-times that of PDGF-AA binding with the same probe aptamer, respectively. Further, fluorescence intensity difference for other biomolecules such as bovine serum album (BSA), ATP and calmodulin was approximately 70 fold smaller when compared to the value obtained for PDGF-BB binding. These results could be explained mainly by the fact that the PDGF-binding aptamer used in this work binds to the three isoforms of PDGF (PDGF-BB, PDGF-AB, and PDGF-AA) with different affinities. Since the target binding aptamer has high specificity toward PDGF-BB, the corresponding reduction in the fluorescence intensity caused by PDGF-AA was clearly lower due to the absence of any binding sites on the aptamer towards PDGF-AA. On the other hand, PDGF-AB protein consists of both A and B chains meaning only one site that could bind to the aptamer. The amino acid sequences of PDGF-A is 60% similar to that of PDGF-B. Therefore, this sensor can detect isoforms with good selectivity. In the other cases where different biomolecules such as BSA, ATP and calmodulin are introduced towards the target binding aptamer, no significant binding is expected due to the unavailability of the binding site and therefore no major relative fluorescence difference was detected. It is noteworthy that although BSA usually contains a high concentration of proteins, it does not affect the selectivity of the probe aptamer used. The excellent selectivity of the sensing platform achieved in this work exhibits the promise of aptamers for cancer biomarker detection. The sensitivity and selectivity of our sensor platform could be even further improved when using high surface area 3D carbon microarrays integrating with functional nanomaterials such as graphene and carbon nanotubes.

Selectivity test of the probe aptamer towards PDGF-BB and other biological components commonly found in blood such as bovine serum albumin, calmodulin and ATP showed a fluorescence intensity difference of approximately 70 fold smaller when compared to the value obtained for PDGF-BB (data not shown). No major relative fluorescence difference was detected for other biomolecules because of their lack of binding with the probe aptamer due to the unavailability of the binding site. The selectivity of the sensing platform achieved in this work towards PDGF-BB compared to other biological components exhibits the promise of aptamers for cancer biomarker detection and for other disorders characterized by the presence of a biomarker in a biological sample at sub-nanomolar concentrations.

In summary, provided herein is highly sensitive detection of an oncoprotein, PDGF, using aptamer/protein binding complex on the 3D carbon microarray platform. For covalent immobilization of the probe aptamer, the carbon surface was bio-functionalized using direct amination technique. The sensor showed a near linear relationship towards protein concentration even in the sub-nanomolar range with excellent selectivity towards other biomolecules. The robust platform of signaling aptamer/protein binding complex on 3D carbon microarrays has the ability to detect wide variety of biomarkers and proteins for potential application in the preliminary diagnosis of cancer.

### REFERENCES

Ellington, A. D., Szostak, J. W., 1990. Nature 346, 818-822
Robertson, D. L., Joyce, G. F. Nature, 1990. 344, 467-468
Tuerk, C., Gold, L., 1990. Science. 249, 505-510
Jayasena, S. D, 1999. Clin Chem. 45, 1628-1650
Shangguan, D., Li, Y., Tang, Z., Cao, Z. C., Chen, H. W., Mallikaratchy, P., Sefah, K., Yang, C. J., Tan, W., 2006. Proc. Natl. Acad. Sci. USA. 103, 11838-11843
Lassila, M., Allen, T. J., Cao, Z., Thallas, V., Jandeleit-Dahm, K. A., Candido, R., Cooper, M. E., 2004. Arterioscler Thromb Vasc Biol. 24, 935-942
Szabó, A., Laki, J., Madsen, H. O., Dósa, E., Prohászka, Z., Rugonfalvi-Kiss, S., Kókai, M., Acsádi, G., Karádi, I., Entz, L., Selmeci, L., Romics, L., Füst, G., Garred, P., 2007. Stroke. 38, 2247-2253
Barst, R. J., 2005. J. Clin. Invest. 115, 2691-2694.
Trojanowska, M., 2008. Rheumatology. 47, v2-v4.
Shih, A. H., Dai, C., Hu, Rosenblum, X. M. H., Koutcher, J. A., Holland, E. C., 2004. Cancer Res. 64, 4783-4789
Yang, L., Fung, C. W., Cho, E. J., Ellington, A. D., 2007. Anal. Chem. 79, 3320-3329
Ruslinda, A. R., Penmatsa, V., Ishii, Y., Tajima, S., Kawarada, H., 2012. Analyst. 137, 1692-1697
Fang, X., Cao, Z., Beck, T., Tan, W., 2001. Anal. Chem. 73, 5752-5757
Fang, X., Sen, A., Vicens, M., Tan, W., 2003. ChemBioChem. 4, 829-834
Vicens, M. C., Sen, A., Vanderlaan, A., Drake, T. J., Tan, W., 2005. ChemBioChem. 6, 900-907
Yang, C. J., Jockusch, S., Vicens, M., Turro, N. J., Tan, W., 2005. Proc. Natl. Acad. Sci. USA. 102, 17278-17283
Jiang, Y., Fang, X., Bai, C., 2004. Anal. Chem. 76, 5230-5235
Zhou, C., Jiang, Y., Hou, S., Ma, B., Fang, X., Li, M., 2006. Anal. Bioanal. Chem. 384, 1175-1180
Huang, C., Chiu, S., Huang, Y., Chang, H., 2007. Anal. Chem. 79, 4798-4804
Huang, C. Chiang, C., Lin, Z., Lee, K., Chang, H., 2008. Anal. Chem. 80, 1497-1504
Huang, C., Huang, Y., Cao, Z., Tan, W., Chang, H., 2005. Anal. Chem. 77, 5735-5741
Lai, R. Y., Plaxco, K. W., Heeger, A. J., 2007. Anal. Chem. 79, 229-233
Degefa, T. H., Kwak, J., 2008, Anal. Chim. Acta. 613, 163-168
Ruslinda, A. R., Tajima, S., Ishii, Y., Ishiyama, Y., Edgington, R., Kawarada, H., 2010. Biosens. and Bioelec. 26, 1599-1604
Phillips, M. F., Lockett, M. R., Rodesch, M. J., Shortreed, M. R., Cerrina, F., Smith, L. M., 2008. Nucleic Acids Res. 36, e7
Ishii, Y., Tajima, S., Kawarada, H., 2011. Appl. Phys. Exp. 4, 027001-3
Wang, C., Taherabadi, L., Madou, M., 2005. IEEE J. Microelectromechanical Systems. 14, 348-358
Ranganathan, S. McCreery, R., Majji, S. M., Madou, M., 2000. J. Electrochem. Soc. 147, 277-282
Singh, A., Jayaram, J., Madou, M., Akbar, S., 2000. J. Electrochem. Soc. 149, E78-E83
Yang, J. H., Penmatsa, V., Tajima, S., Kawarada, H., Wang, C., 2009. Materials Letters. 63, 2680-2683
Xu, H., Malladi, K., Wang, C., Kulinsky, L., Song, M., Madou, M., 2008. Biosens and Bioelec. 23, 1637-1644
Lee, J. A., Hwang, S., Kwak, J., Park, S. I., Lee, S. S., Lee, K. C., 2008. Sens. Actuators B. 129, 372-79
Wang, C., Taherabadi, L., Jia, G., Madou, M., Yeh, Y., Dunn, B., 2004. Electrochemical and Solid-State Letters. 7, A435-A438
Chen, W. Beidaghi, M., Penmatsa, V., Bechtold, K., Kumari, L., Li, W. Z., Wang, C., 2010. Nanotechnology, IEEE Transactions on. 9, 734-739
Penmatsa, V., Yang, J. H., Yu, Y., Wang, C., 2010. Carbon. 48, 4109-4115
Penmatsa, V., Kim, T., Beidaghi, M., Kawarada, H., Wang, Z., Gu, L., Wang, C., Nanoscale. DOI:10.1039/C2NR30161J
Penmatsa, V., Kawarada, H., Wang, C., 2012. J. Micromech. Microeng. 22, 045024-045032
Tuinstra, F., Koenig, J. L., 1970. J. Chem. Phys. 53, 1126-1130
Kawarada, H., Ruslinda, A. R., 2011. Phys. Status Solidi A. 208, 2005-2016

1. A method for detection of a biomarker in a biological sample, comprising
contacting the biological sample with sensor that comprises an aptamer immobilized on a substrate, wherein the aptamer that selectively binds to the biomarker; and wherein the substrate comprises a three dimension (3D) carbon microarray; and wherein the substrate is not a diamond substrate; and
detecting the biomarker in the biological sample by detecting biomarker bound to the sensor.

2. The method of claim 1, wherein the aptamer further comprises a intercalating dye.

3. The method of claim 2, wherein the dye is TOTO.

4. The method of claim 1, that further comprises measuring the biomarker in the biological sample by measuring the amount of biomarker bound to the sensor.

5. The method of claim 4, wherein the measuring of the biomarker comprises measuring a fluorescence signal from the dye, indicative of the amount of the biomarker bound to the aptamer.

6. The method of claim 1, wherein the carbon in the substrate comprises pyrolyzed photoresist carbon.

7. The method of claim 5, wherein the biomarker is a growth factor protein.

8. The method of claim 5, wherein the biomarker comprises a platelet derived growth factor (PDGF) protein.

9. The method of claim 8, wherein the PDGF protein comprises PDGF-B.

10. The method of claim 9, wherein the aptamer comprises the oligonucleotide set forth in SEQ ID NO: 1.

11. The method of claim 1, wherein the biomarker is suspected of being present in the biological sample at a sub-nanomolar concentration.

12. The method of claim 1, wherein the aptamer is covalently attached to the substrate.

13. The method of claim 1, wherein the aptamer comprises a carboxyl modified aptamer and the substrate comprises a 3D carbon microarray modified by direct amination.

14. The method of claim 13, wherein the aptamer is covalently attached to the 3D carbon microarray by an amide bond without the use of a linker molecule.

15. The method of claim 1, wherein the PDGF is present in the biological sample as a dimer.

16. The method of claim 15, wherein biological sample comprises a dimer selected from the group selected from the group consisting of PDGF-AA, PDGF-AB and PDGF-BB.

17. The method of claim 15, wherein the biological sample comprises PDGF-BB.

18. The method of claim 1, wherein the biological sample is blood, serum or plasma.

19. The method of claim 1, wherein the biological sample is from a human subject.

20. The method of claim 1, wherein the human subject has a disorder selected from the group consisting of cancer, atherosclerosis, balloon injury induced restenosis, pulmonary hypertension, organ fibrosis and tumorigenesis.

21. The method of claim 1, wherein the biomarker is present in the sample at a concentration of less than 1 nM.

22. The method of claim 1, wherein the biomarker is present in the sample at a concentration of between 0.005 nM and about 100 nM.

23. A sensor comprising
a three-dimensional (3D) carbon microarray substrate comprising pyrolyzed photoresist carbon; and
an aptamer covalently attached to the substrate, wherein the aptamer selectively binds a biomarker and wherein the aptamer further comprises a intercalating dye.

24-33. (canceled)
